(19) United States
(12) Patent Application Publication
(10) Pub. No.: US 2006/0110281 A1
(43) Pub. Date: May 25, 2006

(54) Scent delivery system

(76) Inventor: Christopher D. Smith, Spring, TX (US)

Correspondence Address: John R. Casperson, PO Box 2174, Friendswood, TX, 77549, US

(21) Appl. No.: 10/993,257

(22) Filed: Nov. 19, 2004

(51) Int. Cl. A61L 9/00

(52) U.S. Cl. 422/5; 422/123

(57) ABSTRACT

The invention provides a method for supplying an aroma to a human bathing area. The method comprises sparging at least one air stream into the bathing area from a location beneath a water surface in the bathing area, and scenting the at least one air stream prior to the sparging step by contacting the air making up the at least one air stream with an aromatic aroma source. The aromatic aroma source can be in the form of a scent delivery module is positioned in the air flow path to supply bubbles to the pool or tub. One embodiment of the module comprises sidewall means defining an inlet, an outlet, and a gas flow path for a gas stream extending between the inlet and the outlet. The gas flow path is divided along a portion of its length into a first flow path and a second flow path. A first portion of the gas stream flows through the first flow path and a second portion of the gas stream flows through the second flow path. The first gas flow path and the second gas flow path are rejoined for a combined flow path through the outlet. The sidewall means further comprises a vaporizer chamber positioned in the second flow path for introducing scent vapors into the second portion of the gas stream.

The invention relates to delivering aromas, scents, and gases sub-aquatically for human enjoyment and/or therapy in, for example, bathtubs, spas and hot tubs.

### BACKGROUND OF THE INVENTION

In a bathing environment, scents have traditionally been delivered via candles and incense placed around the bathroom and by other types of oils and capsules used in the bath water. Sometimes, aerosols are used.

The candles and incense may constitute a fire hazard, and, due to particulate emissions, a health hazard. The oils and capsules can ruin delicate pool surfaces, particularly concrete spas and they also leave heavy residues in the water. They further only deliver a certain scent in a selected area of the spa or bathtub. The aerosols are generally not long-lasting.

A system to replace the traditional scent delivery means that are now on the market would be very desirable.

Many people know that when a gas is released under water it rises to the surface, and its scent is intensified as it hits the water surface, thereby creating a more concentrated smell. A scent delivery system which takes advantage of this phenomenon would create a more intense experience, and would be very desirable. A scent delivery system which masks the smell of chlorine and other disinfectants (also enhanced in spas and Jacuzzi tubs) would be even further desirable.

Spas and hot tubs provide physical therapy through two mechanisms, increased pressure through a series of evenly placed air and waterjets, and heated water. These actions create air bubbles and temperatures in the range of 95 degrees up to 102 degrees. A system to combine spa therapy with aroma therapy would be very desirable.

### OBJECTS OF THE INVENTION

It is an object of this invention to provide new and innovative delivery systems for various aroma therapy combinations. These systems take advantage of the notion of delivering aromas, scents, and gases sub-aquatically, in bathtubs, Jacuzzi tubs and spas. Injecting scents into this environment through air jets creates an intense and pleasurable olfactory experience for the user. The scent is carried to the surface inside the air bubbles, the bubbles pop on the surface creating steam, and the steam then carries the scent into the nose of the user. The scent is intensified by the hot water. Also residual material is minimized because most scents will not solubilize with the water. This idea creates a new and effective way to administer new aroma therapy techniques inside bath tubs and spas.

This new and innovative approach to sub-aquatic scent delivery will provide a new and safe method for all aromas and spa therapy needs. It will change how solutions are manufactured and packaged. The bathroom industry will be ever-changed. In lieu of the traditional essential oils and candles (which have always been a fire hazard) a potential user can enjoy a more effective way to deliver scents in the bathroom and in the hot tub, while improving a general state of well-being. Scent delivery under water has not been looked at in this way, nor has there been a line of products created to facilitate the idea.

### SUMMARY OF THE INVENTION

One embodiment of the invention provides a method for supplying an aroma to a human bathing area. The bathing area contains water. The method comprises sparging at least one air stream into the bathing area from a location beneath a water surface in the bathing area, and scenting the at least one air stream prior to the sparging step by contacting the air making up the at least one air stream with an aromatic aroma source.

In another embodiment of the invention, there is provided a pool system. The system is formed by a sidewall at least partially defining the pool or tub. A plurality of nozzles open through the sidewall. A manifold is connected to the nozzles. An air blower is connected to the manifold, so that an air flow path is defined from the atmosphere to the pool or tub via the air blower and the manifold. A scent delivery module is positioned in the air flow path to supply scent to the pool or tub.

Another embodiment of the invention provides a scent delivery module which is well suited for installation in new or preexisting pools or spas. The module comprises a sidewall means defining an inlet, an outlet, and a gas flow path for a gas stream extending between the inlet and the outlet. The gas flow path is divided along a portion of its length into a first flow path and a second flow path. A first portion of the gas stream flows through the first flow path and a second portion of the gas stream flows through the second flow path. The first gas flow path and the second gas flow path are rejoined for a combined flow path through the outlet. The sidewall means further defines a vaporizer chamber positioned in the second flow path for introducing scent vapors into the second portion of the gas stream.

### BRIEF DESCRIPTION OF THE DRAWINGS

FIG. 1 is a pictorial illustration of a scent delivery module according to one embodiment of the invention.

FIG. 2 is a side view of the module shown in FIG. 1 when viewed along lines 2-2. Certain features are shown in ghost or by break-away.

FIG. 3 is a side view of another embodiment of the invention.

FIG. 4 is an illustration showing a use of the FIG. 2 invention.

FIG. 5 is an illustration, partly schematic, of another embodiment of the invention.

FIG. 6 is an end view of a scent module portion of the FIG. 5 invention.

FIG. 7 is a cross-sectional view of the module shown in FIG. 6 taken along lines 7-7.

FIG. 8 is an exploded view of the module shown in FIG. 7.

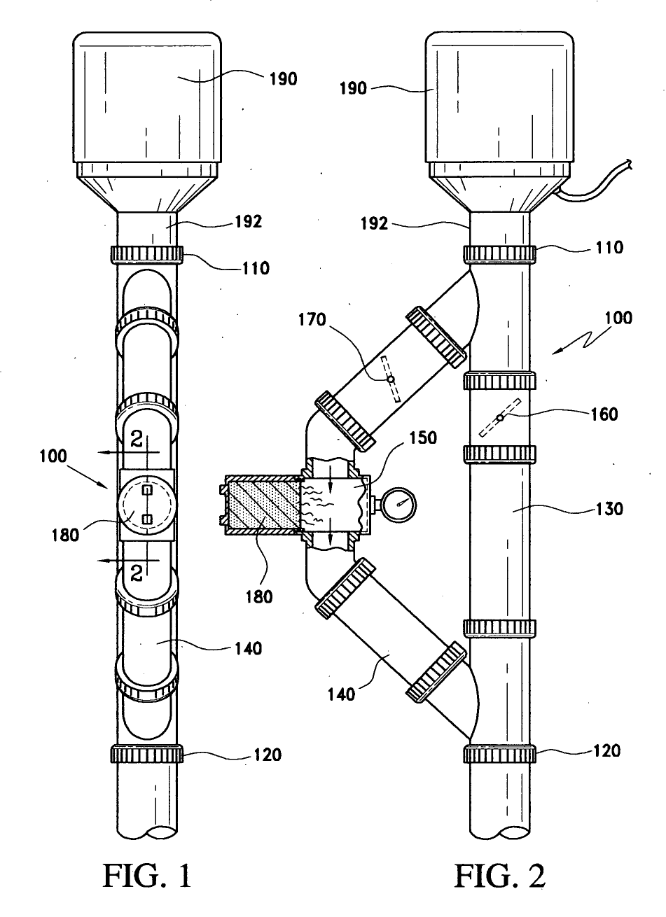

### DETAILED DESCRIPTION OF THE INVENTION

Literally thousands of scents can be used in the invention. Common aromatherapy scents include angelica, basil, benzoin, bergamot, white birch, black pepper, borneol, cade, cajeput, cardomon, carrot seed, cassie, Canadian balsam, cedarwood, chamomile, citronella, Cary sage, clove, coriander, cypress, elem., eucalyptus, fennel, frankincense, geranium, ginger, grapefruit, helichrysum, jasmine, juniper, lavender, lemon, marjoram, melissa, myrrh, neroli, nutmeg, orange, palmarosa, patchouli, peppermint, pine, rose, rosemary, sandalwood, tea tree, thyme, vetiver, ylang-ylang. However, the invention can be used with flower scents, for example, rose, magnolia, orange blossom, lavender and jasmine, woods scents such as acacia, pine, holly or walnut, perfume and cologne scents such as Chanel No. 5™, Musk™, Obsession™ and Opium™, herb and plant scents such as clover, new mown hay, wintergreen, Spanish moss; miscellaneous scents such as baby powder, Downy™, Irish Spring™, and tobacco, holiday scents such as balsam fir, cinnamon, eggnog and pumpkin, and food and drink scents such as ambrosia, brownie, rum, wine. For therapeutic uses, for example, treatment of asthma, angelica, basil, Canadian balsam, benzoin, cajeput, clary sage, clove bud, cypress, eucalyptus, fennel frankincense, helichrysum, lavender, lemon, marjoram, melissa, myrrh, peppermint, pine, rose, rosemary, tea tree, and thyme can be used. For suppression of coughing, angelica, basil, benzoin, black pepper, borneol, cajeput, Canadian balsam, cedarwood, clary sage, cypress, elemi, eucalyptus, frankincense, ginger, helichrysum, juniper, marjoram, myrrh, rose, rosemary, scotch pine, and tea tree can be used. The above described scents are intended as exemplary, rather than limiting, of the scents that can be used in the invention. They are all commercially available in various forms, the form of concentrated oils being the most common.

The carrier for the scent can be selected from a wide variety of materials. For example, the scent can be carried by a wax bar, or in wax pellets. Or the scent can be carried by a fibrous material, which can be of natural or synthetic origin. For example, natural sponge and Spanish moss are suitable natural carriers. Paper, woven and nonwoven cloths, and plastic or glass beads can be used as well. Some scents, such as certain herbal scents, can be used in chipped, ground or comminuted form without a separate carrier. The intensity of the scent can be adjusted as desired in a preferred embodiment of the invention by regulating the flow of air over the source of the scent as hereinafter described.

One embodiment of the invention provides a scent delivery module 100 which is well suited for installation in new or pre-existing pools or spas. The module comprises sidewall means defining an inlet 110, an outlet 120, and a gas flow path for a gas stream extending between the inlet and the outlet. The gas flow path is divided along a portion of its length into a first flow path 130 and a second flow path 140. A first portion of the gas stream flows through the first flow path and a second portion of the gas stream flows through the second flow path. The first gas flow path and the second gas flow path are rejoined for a combined flow path through the outlet. The sidewall means further defines a vaporizer chamber 150 positioned in the second flow path for introducing scent vapors into the second portion of the gas stream.

The just-described module is easily installed in existing units by cutting the existing air-in line and inserting the module with suitable couplings. Preferably, the module is installed so that the vaporizer chamber, as well as the blower, are above the water level in the pool or tub unit.

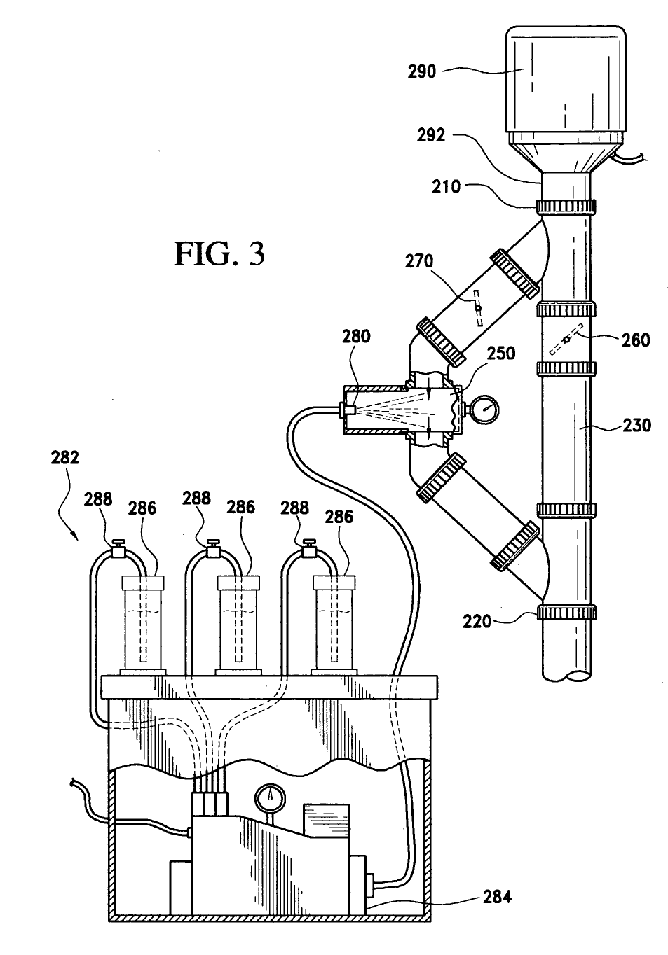

In the FIG. 2 embodiment, the scent delivery module comprises a first externally adjustable valve 160 positioned in the first flow path to regulate flow of the first portion of the gas stream, and a second externally adjustable valve 170 positioned in the second flow path to regulate flow of the second portion of the gas stream. In the FIG. 3 embodiment, valves 260 and 270 perform the function. The valves provide a mechanism to adjust the intensity of the scent. For example, the scent of wood chips positioned in the vaporizer chamber would diminish in intensity over time, and this could be at least partially compensated for by adjusting the valves to provide for a greater portion of the air flow to be through the vaporizer chamber.

In a preferred embodiment, a means is provided for positioning the aromatic material in the vaporizer chamber. For example, the means could be a wick (which could draw scented liquid from a holding chamber), a pad, such as a rolled up Scotchbrite™ pad which is saturated prior to use with concentrated scent, a bar, such as a bar of scented wax or soap, a bed of scented pellets or chips, or a particle bed, such as a bed of ground spices. For convenience, it is preferred that the scent carrier be contained in a canister 180 which, during use, is swept by the gas flowing along the second flow path. A canister is desirable because a canister can be easily removed from the module and replaced without requiring a clean up. More preferably, the canister is generally cylindrically shaped, and is received in a port defined next to the vaporizer chamber, for example, threadably received.

A nozzle 280 provides an alternative means for positioning the aromatic material in the vaporizer chamber and is shown in FIG. 3. In this embodiment, a source 282 of aromatic material in liquid form is positioned in flow communication with the nozzle. A pump 284 draws the liquid from a source and forces it through the nozzle to form a spray which is injected into the second flow path. If desired, a plurality 286 of sources of liquid aromatic material are positioned in flow communication with an inlet to the pump. The sources each include a valve means to permit a selected liquid aromatic material to flow to the pump.

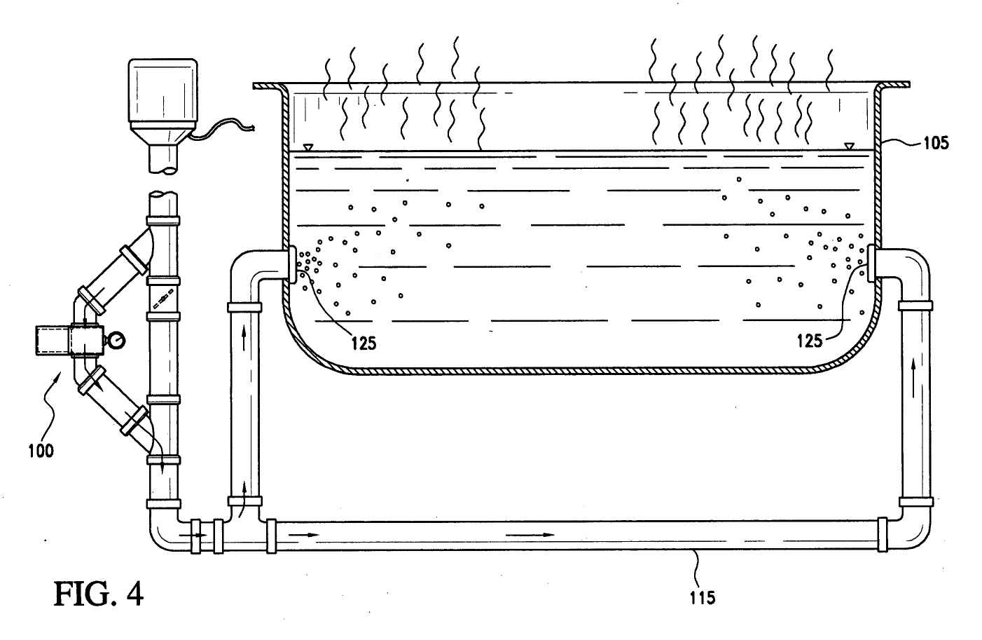

For use, the scent delivery module is combined with a blower, for example, a blower 190 as shown in FIG. 2, or a blower 290 as shown in FIG. 3. A conduit, for example a conduit 192 as shown in FIG. 3 or a conduit 292 as shown in FIG. 3, connects an outlet of the blower to an inlet of the sidewall means. There is provided a sidewall at least partially defining a pool or tub, for example, sidewall 105 in FIG. 4. A manifold 115 connects the outlet of the sidewall means with the pool or tub. The manifold opens through the sidewall defining the pool or tub to inject the scented bubbles into the bath.

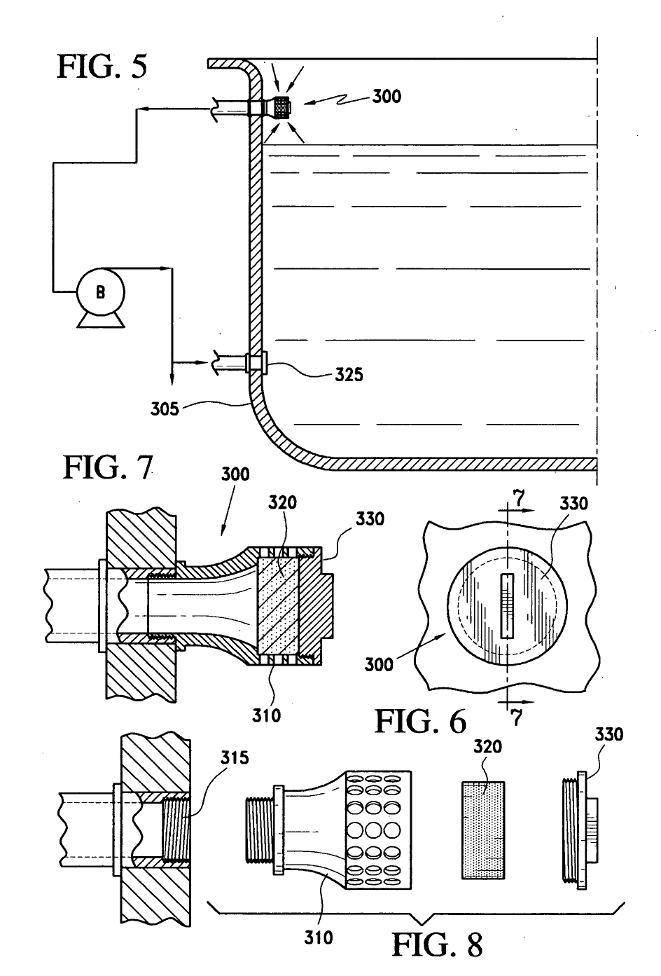

In another embodiment of the invention, there is provided a pool system. The system is formed by a sidewall at least partially defining the pool or tub, for example, sidewall 105 in FIGS. 4 and 305 in FIG. 5. A plurality of nozzles open through the sidewall. For example nozzles 125 in FIG. 4 and nozzle 325 in FIG. 5. A manifold is connected to the nozzles. For example, manifold 115 in FIG. 4 and as shown schematically in FIG. 5. An air blower is connected to the manifold, so that an air flow path is defined from the atmosphere to the pool or tub via the air blower and the manifold. A scent delivery module is positioned in the air flow path to supply scent to the pool or tub. For example, module 100 in FIG. 4 or module 300 in FIG. 5.

In the embodiment shown in FIGS. 5-8, the air blower has an inlet and an outlet. An air inlet conduit has one end connected to the blower inlet, and an open end 315 (See FIG. 8). The open end is typically positioned in the tub above the waterline. The scent delivery module 300 is positioned over the open end. The scent delivery module preferably comprises a perforated housing 310 having a first end and a second end. The first end of the scent delivery module is connected to the open end of the air conduit. A scent element 320 is positioned in the housing.

For this application, the scent element is preferably selected from a wick, a pad, a bar, a pellet or a particle which carries an aromatic material and is contained in a perforated generally cylindrical sidewall portion of the housing. To permit replenishment of the scent element, the module preferably includes a removable end closure 330 positioned on the second end of the housing. For convenience, the end closure is threadably connected to the second end of the housing and the housing is threadably connected to the open end of the air conduit. In the illustrated embodiment, the second end of the housing is of greater diameter than the first end of the housing and the scent element is generally cylindrically shaped.

In another embodiment, the invention provides a method for supplying an aroma to a human bathing area. The bathing area contains water. The method comprises sparging at least one air stream into the bathing area from a location beneath a water surface in the bathing area, and scenting the at least one air stream prior to the sparging step by contacting the air making up the at least one air stream with an aromatic aroma source.

Preferably the water is heated. More preferably, it is further maintained at a temperature within the range of 95 to 102 degrees F.

The aromatic source can be carried on a fibrous or other material as previously described, and further provided in the form of an easily replaceable canister, as well. It can also be sprayed into the air stream, or employed in the other forms discussed hereinabove.

Examples of aromatic aroma sources include menthol, eucalyptus, and aloe, and chamomile and sage. Aromatherapy scents can also be used, for example the aromatic aroma source can be an aromatic substance is selected from the group consisting of angelica, basil, benzoin, bergamot, white birch, black pepper, borneol, cade, cajeput, cardomon, carrot seed, cassie, Canadian balsam, cedarwood, chamomile, citronella, clary sage, clove, coriander, cypress, elemi, eucalyptus, fennel, frankincense, geranium, ginger, grapefruit, helichrysum, jasmine, juniper, lavender, lemon, marjoram, melissa, myrrh, neroli, nutmeg, orange, palmarosa, patchouli, peppermint, pine, rose, rosemary, sandalwood, tea tree, thyme, vetiver, and ylang-ylang.

For therapeutic purposes, for example, the treatment of asthma, angelica, basil, Canadian balsam, benzoin, cajeput, clary sage, clove bud, cypress, eucalyptus, fennel, frankincense, helichrysum, lavender, lemon, marjoram, melissa, myrrh, peppermint, pine, rose, rosemary, tea tree, and thyme can be used. For suppression of coughing, angelica, basil, benzoin, black pepper, borneol, cajeput, Canadian balsam, cedarwood, clary sage, cypress, elemi, eucalyptus, frankincense, ginger, helichrysum, juniper, marjoram, myrrh, rose, rosemary, scotch pine, and tea tree can be used.

This new aroma therapy delivery technique can be applied to three major industries. The first and most important is the medical field. Products such as Vick's™ (vapor rub), Mentholatum™, Breathe Right Inc., and Icy Hot™ can be readily adapted as inserts to fit the scent delivery canisters. This will be a new and effective way to deliver certain medicinal needs that improve congestion and breathing problems. Many hospitals utilize spas in their physical therapy processes. Combining Aloe Vera, Eucalyptus, and Menthol in these units will give new diversities to expand medical spa and physical therapy unit treatments

The second major industry is the cosmetic industry. The various essentials on the market can be utilized in our units to replace the many bath crystals and oils placed in the water manually which leave heavy residues in the water.

The third industry is the ever-growing aroma therapy industry. This industry has traditionally delivered scents through incense, candles, bath crystals, and aerosol sprays. The invention will create a new market in sub-aquatic scent delivery systems for the in-home bath and hot tub market.

The biggest benefactor to this new delivery system will be the pool and spa industry. There are literally millions of fiberglass and concrete pools and spas in the USA. By injecting into the air intake/blower systems in our devices, the scent will be evenly and better distributed. Also by blowing the scents into the air bubble systems, the scent is captured into the bubbles and carried to the top of the water's surface where it pops and the scent is released evenly. Also since heat plays a big part in intensifying the scent, the steam from the heated water will also carry the scent into the user's olfactory system, providing a more concentrated scent.

There are three products I have invented to deliver these types of scents into a bathtub or spa environment: the In-Home Spa Tub Unit, and Two Outdoor Spa Units.

The In-Home Spa Tub Unit is designed to replace the air adjustment knob for all spa tubs. It has a scent bar container that is inter-changeable, and still allows air flow adjustment to the bubbles. I will provide converters to fit the different array of diameters and threading sizes for the many brands of Jacuzzi tubs on the markets. The replaceable units contain a canister to house the scent, with vented threads to allow the scent and air to combine prior to entering the pre existing multi-jet piping system. The air is sucked through the scent canister which picks up the scent and then delivered under the water and out the various jets in the spa tub, the bubbles and steam deliver the scent to the user.

There are two outdoor units, one is an inline replacement module that diverts the main airflow from the spa blower into a scent canister with a special filter element that contains the scent, the air is then routed back into the primary blower pipe which evenly distributes the scent through the many jets in the hot tub. There are valves to control the amount of scented air injected into the spa jets The filter elements are interchangeable to create the different scent environments. Aroma therapy is combined with traditional spa therapy to create a more satisfied environment.

The second outdoor unit is a mist/gas injection system that combines the above air diverter and a 110-volt high pressure pump that is in line with three separate canisters. The three canisters have calibrated valves to control the amount of solution to be misted into the canister. There is a relay on the pump that controls pump duration mist released into the line. A remote is also added so one can push the remote control to activate programmed pump duration into the lines. This allows one to combine elements such as: menthol, eucalyptus, and aloe to create a more comprehensive aroma therapy combination. It also creates a more intense aroma of these elements due the delivery of these under water into the air bubbles and steam of the system. This system gives the user more options on combinations to create the desired aroma therapy technique. It also allows the user to control the amount of mist injected into the spa by controlling pump duration and also by using the remote control device. The mist also provides more gas into the spa depending on which type of gas or mist is injected into the water. This system can provide medicinal needs, cosmetic needs, and aroma therapy needs.

While certain preferred embodiments of the invention have been described herein, the invention is not to be constructed as being so limited, except to the extent that such limitations are found in the claims.

What is claimed is:

1. A method for supplying an aroma to a human bathing area, said human bathing area containing water, said method comprising
sparging at least one air stream into the bathing area from a location beneath a water surface in the bathing area, and
scenting said at least one air stream prior to the sparging step by contacting the air making up said at least one air stream with an aromatic aroma source.

2. A method as in claim 1 further comprising maintaining the temperature of the water within the range of 95 to 102 degrees F.

3. A method as in claim 2 wherein the aromatic aroma source is contained in a canister.

4. A method as in claim 2 wherein the aromatic aroma source is sprayed into the at least one air stream.

5. A method as in claim 2 wherein the aromatic aroma source is an aromatic substance is selected from the group consisting of menthol, eucalyptus, and aloe.

6. A method as in claim 2 wherein the aromatic aroma source is an aromatic substance is selected from the group consisting of angelica, basil, benzoin, bergamot, white birch, black pepper, borneol, cade, cajeput, cardomon, carrot seed, cassie, Canadian balsam, cedarwood, chamomile, citronella, clary sage, clove, coriander, cypress, elemi, eucalyptus, fennel, frankincense, geranium, ginger, grapefruit, helichrysum, jasmine, juniper, lavender, lemon, marjoram, melissa, myrrh, neroli, nutmeg, orange, palmarosa, patchouli, peppermint, pine, rose, rosemary, sandalwood, tea tree, thyme, vetiver, ylang-ylang.

7. A pool system comprising
a sidewall at least partially defining a pool or tub,
a plurality of nozzles opening through the sidewall,
a manifold connected to the nozzles,
an air blower connected to the manifold, an air flow path being defined from the atmosphere to the pool or tub via the air blower and the manifold, and
a scent delivery module positioned in the air flow path to supply scent to the pool or tub.

8. A system as in claim 7 wherein the air blower has an inlet and an outlet, and an air inlet conduit having one end connected to the blower inlet, and an open end, and the scent delivery module is positioned over the open end.

9. A system as in claim 8 wherein the scent delivery module comprises
a perforated housing having a first end and a second end, and
a scent element positioned in the housing, the first end of the scent delivery module being connected to the open end of the air conduit.

10. A system as in claim 9 wherein the scent element comprises
a wick, a pad, a bar, a pellet or a particle which carries an aromatic material and is contained in a perforated generally cylindrical sidewall portion of the housing.

11. A scent delivery module comprising
a sidewall means defining an inlet, an outlet, and a gas flow path for a gas stream extending between the inlet and the outlet, said gas flow path being divided along a portion of its length into a first flow path and a second flow path, a first portion of the gas stream flowing through the first flow path and a second portion of the gas stream flowing through the second flow path, said first gas flow path and said second gas flow path being rejoined for a combined flow path through the outlet, said sidewall means further defines a vaporizer chamber positioned in the second flow path for introducing scent vapors into the second portion of the gas stream.

12. A scent delivery module as in claim 11 further comprising
a first externally adjustable valve positioned in the first flow path to regulate flow of the first portion of the gas stream, and
a second externally adjustable valve positioned in the second flow path to regulate flow of the second portion of the gas stream.

13. A scent delivery module as in claim 11 further comprising
a means for positioning an aromatic material in the vaporizer chamber.

14. A scent delivery module as in claim 13 wherein the means for positioning comprises a canister.

15. A scent delivery module as in claim 13 wherein the means for positioning includes a nozzle.

16. A scent delivery module as in claim 15 wherein the means for positioning further comprises a source of aromatic material in liquid form in flow communication with the nozzle.

17. A scent delivery module as in claim 16 further comprising a pump to draw liquid aromatic material from the source and force the liquid aromatic material through the nozzle to form a spray.

18. A scent delivery module as in claim 17 further comprising
a plurality of sources of liquid aromatic material positioned in flow communication with an inlet to the pump, said sources each including valve means to permit a selected liquid aromatic material to flow to the pump.

19. A scent delivery module as in claim 13 further comprising, in combination
a blower, and
a conduit connecting an outlet of the blower to an inlet of the sidewall means.

20. A scent delivery module as in claim 19 further comprising, in combination
a sidewall at least partially defining a pool or tub, and
a manifold connecting the outlet of the sidewall means with the pool or tub, said manifold opening through the sidewall defining the pool or tub.
